(19) United States
(12) Patent Application Publication
(10) Pub. No.: US 2005/0204811 A1
(43) Pub. Date: Sep. 22, 2005

(54) System and method for measuring flow in implanted cerebrospinal fluid shunts

(76) Inventor: Samuel R. Neff, Rosemont, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/113,758

(22) Filed: Apr. 25, 2005

Related U.S. Application Data
(63) Continuation-in-part of application No. 10/770,754, filed on Feb. 3, 2004.

(51) Int. Cl. A61M 5/00

(52) U.S. Cl. 73/204.11; 73/204.23; 604/9

(57) ABSTRACT

A system and method for a thermal convection flow detection in a cerebrospinal fluid shunt that uses very little power for extended operation and for providing flow data to a remotely-located device.

### BACKGROUND OF THE INVENTION

1. Field of Invention

This invention relates to cerebrospinal fluid shunts and, more particularly, to apparatus and methods for quantitatively detecting the flow of cerebrospinal fluid in such shunts non-invasively.

2. Description of Related Art

Hydrocephalus, a common disease, is caused by failure of normal circulation of the cerebrospinal fluid (CSF). Normally, cerebrospinal fluid is made in the brain (at a rate of 0.03 ml/minute), circulates through pathways in the brain, and is then absorbed into the bloodstream. In hydrocephalus the production of CSF continues normally, but the circulation and/or absorption are impaired. The result of this imbalance between the production and absorption is fluid accumulation in the brain, excessive head growth (in children), and brain deformation. Untreated, this leads to brain damage and death.

Hydrocephalus is commonly treated by surgically implanting a plastic tube (a “shunt”) under the skin. One end of the tube is implanted in the brain and one end in another part of the body. The unabsorbed CSF is diverted by this tube from the brain to another site (such as a vein) where it can be absorbed by natural processes. Between 25,000 and 50,000 such surgical procedures are performed yearly, in the United States alone.

Although life-saving, shunts have a failure rate of 60%. Failures are treated surgically by replacing the clogged or broken portions of the shunt.

Once the shunt malfunctions, there is a window of only a few days or even hours before irreparable brain damage occurs. This is an important clinical problem in neurosurgery, because the key symptoms of shunt malfunction-headache, nausea, and vomiting—are also symptoms of many other diseases. Shunt malfunctions can be detected by looking for early brain deformation with a CT scan or MRI, but it is obviously impractical to perform such an expensive study ($300-$1200) every time a patient has a headache.

The following describe different apparatus and methodologies that have been used to monitor, determine or treat body fluid flow, including CSF flow through a shunt.

“A Thermosensitive Device for the Evaluation of the Patency of Ventriculo-atrial Shunts in Hydrocephalus”, by Go et al. (Acta Neurochirurgica, Vol. 19, pages 209-216, Fasc. 4) discloses the detection of the existence of flow in a shunt by placement of a thermistor and detecting means proximate the location of the shunt and the placement of cooling means downstream of the thermistor. The downstream thermistor detects the cooled portion of the CSF fluid as it passes from the region of the cooling means to the vicinity of the thermistor, thereby verifying CSF flow. However, among other things, the apparatus and method disclosed therein fails to teach or suggest an apparatus/method for quantifying the flow of the fluid through the shunt.

In “A Noninvasive Approach to Quantitative Measurement of Flow through CSF Shunts” by Stein et al., Journal of Neurosurgery, 1981, April; 54(4):556-558, a method for quantifying the CSF flow rate is disclosed. In particular, a pair of series-arranged thermistors is positioned on the skin over the CSF shunt, whereby the thermistors independently detect the passage of a cooled portion of the CSF fluid. The time required for this cooled portion to travel between the thermistors is used, along with the shunt diameter, to calculate the CSF flow rate.

See also “Noninvasive Test of Cerebrospinal Shunt Function,” by Stein et al., Surgical Forum 30:442-442, 1979; and “Testing Cerebropspinal Fluid Shunt Function: A Noninvasive Technique,” by S. Stein, Neurosurgery, 1980 June 6(6): 649-651. However, the apparatus/method disclosed therein suffers from, among other things, variations in thermistor signal due to environmental changes.

U.S. Pat. No. 4,548,516 (Helenowski) discloses an apparatus for indicating fluid flow through implanted shunts by means of temperature sensing. In particular, the apparatus taught by Helenowski comprises a plurality of thermistors mounted on a flexible substrate coupled to a rigid base. The assembly is placed on the skin over the implanted shunt and a portion of the fluid in the shunt is cooled upstream of the assembly. The thermistors detect the cooled portion of the fluid as it passes the thermistor assembly and the output of the thermistor is applied to an analog-to-digital converter for processing by a computer to determine the flow rate of the shunt fluid.

U.S. Pat. No. 6,413,233 (Sites et al.) discloses several embodiments that utilize a plurality of temperature sensors on a patient wherein a body fluid (blood, saline, etc.) flow is removed from the patient and treated, e.g., heated or cooled, and then returned to the patient. See also U.S. Pat. No. 5,494,822 (Sadri). U.S. Pat. No. 6,527,798 (Ginsburg et al.) discloses an apparatus/method for controlling body fluid temperature and utilizing temperature sensors located inside the patient's body.

U.S. Pat. No. 5,692,514 (Bowman) discloses a method and apparatus for measuring continuous blood flow by inserting a catheter into the heart carrying a pair of temperature sensors and a thermal energy source. See also U.S. Pat. No. 4,576,182 (Normann).

U.S. Pat. No. 4,684,367 (Schaffer et al.) discloses an ambulatory intravenous delivery system that includes a control portion of an intravenous fluid that detects a heat pulse using a thermistor to determine flow rate.

U.S. Pat. No. 4,255,968 (Harpster) discloses a fluid flow indicator which includes a plurality of sensors placed directly upon a thermally-conductive tube through which the flow passes. In Harpster a heater is located adjacent to a first temperature sensor so that the sensor is directly within the sphere of thermal influence of the heater.

U.S. Pat. No. 3,933,045 (Fox et al.) discloses an apparatus for detecting body core temperature utilizing a pair of temperature sensors, one located at the skin surface and another located above the first sensor wherein the output of the two temperature sensors are applied to a differential amplifier heater control circuit. The control circuit activates a heat source in order to drive the temperature gradient between these two sensors to zero and thereby detect the body core temperature.

U.S. Pat. No. 3,623,473 (Andersen) discloses a method for determining the adequacy of blood circulation by measuring the difference in temperature between at least two distinct points and comparing the sum of the detected temperatures to a reference value.

U.S. Pat. No. 3,762,221 (Coulthard) discloses an apparatus and method for measuring the flow rate of a fluid utilizing ultrasonic transmitters and receivers.

U.S. Pat. No. 4,354,504 (Bro) discloses a blood-flow probe that utilizes a pair of thermocouples that respectively detect the temperature of a hot plate and a cold plate (whose temperatures are controlled by a heat pump. The temperature readings are applied to a differential amplifier. Energization of the heat pump is controlled by a comparator that compares a references signal to the differential amplifier output that ensures that the hot plate does not exceed a safety level during use.

Thus, in view of the foregoing, there remains a need for a method and system for detecting shunt flow non-invasively, using low power and without damaging white blood cells, thereby allowing the patient and physician to safely, easily and economically determine shunt function.

All references cited herein are incorporated herein by reference in their entireties.

### BRIEF SUMMARY OF THE INVENTION

A system for detecting the flow rate of cerebrospinal fluid (CSF) in a CSF shunt implanted inside a living being. The system comprises: a flow detector that is momentarily powered to generate a thermal heat pulse in the CSF flow and whose movement over time therein is detected using a pair of temperature sensors (e.g., thermistors). The flow detector further comprises a processor (e.g., a microcontroller) for determining a CSF flow rate from the detected movement and wirelessly transmits a signal representative of the determined CSF flow rate; an activator (e.g., a magnet, a RF transmitter, an IR transmitter, an ultrasonic transmitter, etc.), external to the living being, that causes the flow detector to be momentarily powered; and a remotely-located receiver for receiving the signal representative of the determined CSF flow rate.

A method for detecting the flow rate of cerebrospinal fluid (CSF) in a CSF shunt implanted inside a living being. The method comprises the steps of: generating a heat pulse at a predetermined location along or within the CSF shunt; detecting at least one temperature value of the CSF flow upstream of the predetermined location and detecting at least one temperature value of the CSF flow downstream of the predetermined location; obtaining a maximum temperature difference value between the at least one upstream temperature value and the at least one downstream temperature value; relating the maximum temperature difference value to a known CSF flow rate to determine the CSF flow rate; and wirelessly transmitting the determined CSF flow rate to a remote location.

### BRIEF DESCRIPTION OF SEVERAL VIEWS OF THE DRAWINGS

The invention will be described in conjunction with the following drawings in which like reference numerals designate like elements and wherein:

FIG. 1 is a functional diagram of the system and method of the present invention;

FIG. 2 is schematic view of the shunt depicting the temperature sensor pair and heating element of the embedded flow detector and of an alternative externally-located flow detector;

FIG. 3 is a circuit schematic of the flow detector, microcontroller and supporting electronics;

FIG. 3A is an alternative circuit schematic of the flow detector, microcontroller and supporting electronics;

FIG. 4A is a graph of test results for various flow rates of CSF fluid with the difference in temperature between the upstream temperature sensor and the downstream temperature sensor (ΔT) versus time based on a heating pulse;

FIG. 4B is a plot of the relationship between the temperature difference (ΔT) sensed by the flow detector and the actual flow;

FIG. 4C is an exemplary look-up table based on the temperature data of FIGS. 4A-4B that is used by the microcontroller in determining the CSF flow rate;

FIG. 5 is a flow chart of the microcontroller operation; and

FIG. 6 is an exemplary remote detector for the flow rate data transmitted from the flow detector for display at the remote location.

### DETAILED DESCRIPTION OF THE INVENTION

The present invention provides an apparatus and method for using pulsed heating to automatically detect CSF flow while using very little power and while raising the temperature of the CSF flow in the vicinity of the apparatus less than 1° C., thereby minimizing any damage to white blood cells that could result in clogging the shunt, immune reactions or other patient injuries.

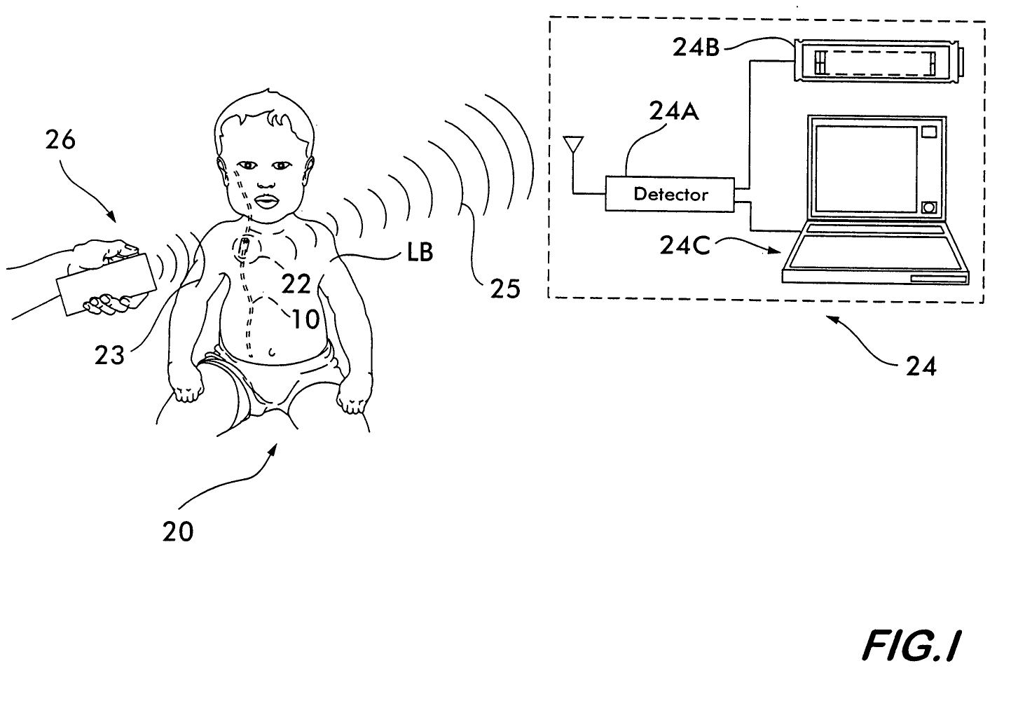

As shown in FIG. 1, the present invention 20 comprises a cerebrospinal fluid shunt 10 having a flow detector 22, a remotely-located receiver/display 24 (e.g., a detector 24A, a display 24B or a computer such as a laptop 24C, etc.) and a remotely-located activator 26.

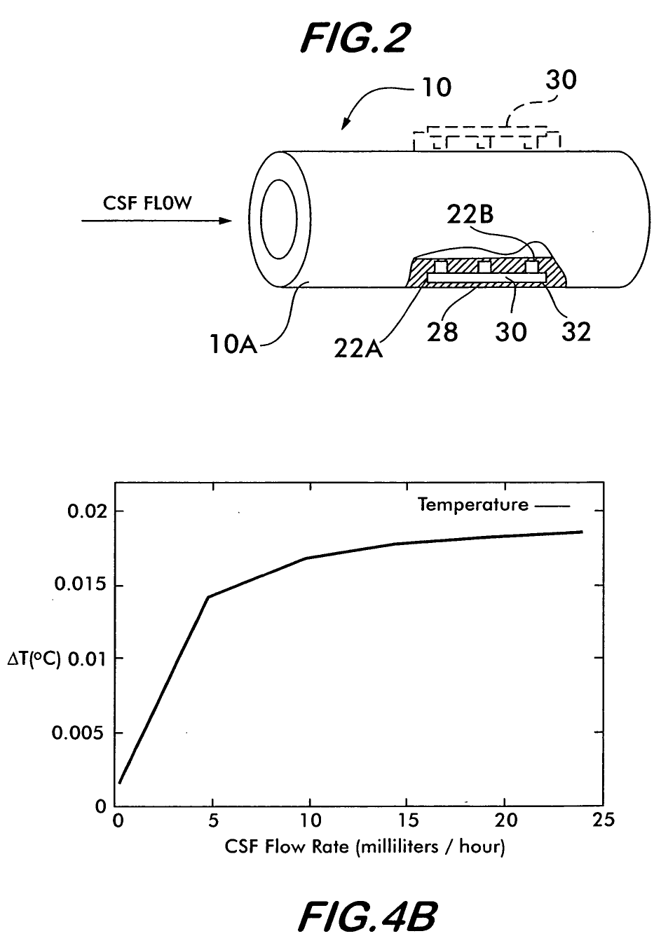

In particular, the cerebrospinal fluid shunt 10 comprises tubing (e.g., plastic (e.g., silicone), or ceramic, metal, etc.) which is disposed inside a living being LB. The flow detector 22 is preferably embedded within the wall 10A of the shunt 10 as shown in FIG. 2. Alternatively, the flow detector 22 can be located in other locations such as, but not limited to, the outside surface of the shunt 10. The flow detector 22 comprises a pair of temperature sensors (e.g., thermistors) 22A and 22B and a heating element (e.g., a resistor, chip resistor, etc.) 28 and a microcontroller 30 (e.g., Atmel Corporation: Attiny 15L) along with supporting electronics 32. It should be understood that if the temperature sensors 22A/22B and the heating element 28 are embedded within the wall of the shunt, the location of the microcontroller 30 and the supporting electronics 32 is not required to also be within the wall 10A of the shunt 10 but with integrated circuit design fabricating methods, it would be within the broadest scope of the invention to have these components also embedded within the wall 10A of the shunt 10. The temperature sensors 22A/22B are displaced from each other along the length of the shunt 10 with the heating element 28 positioned between the two sensors 22A/22B. Testing has determined that a preferable spacing between each temperature sensor 22A/22B and the heating element 28 is approximately 2 mm.

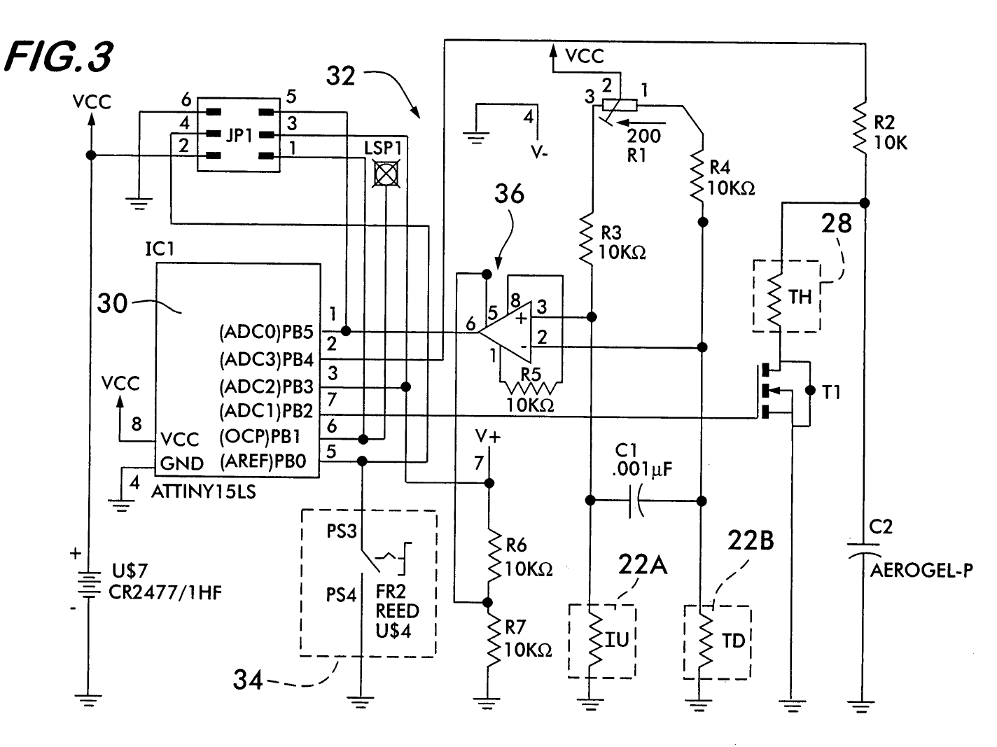

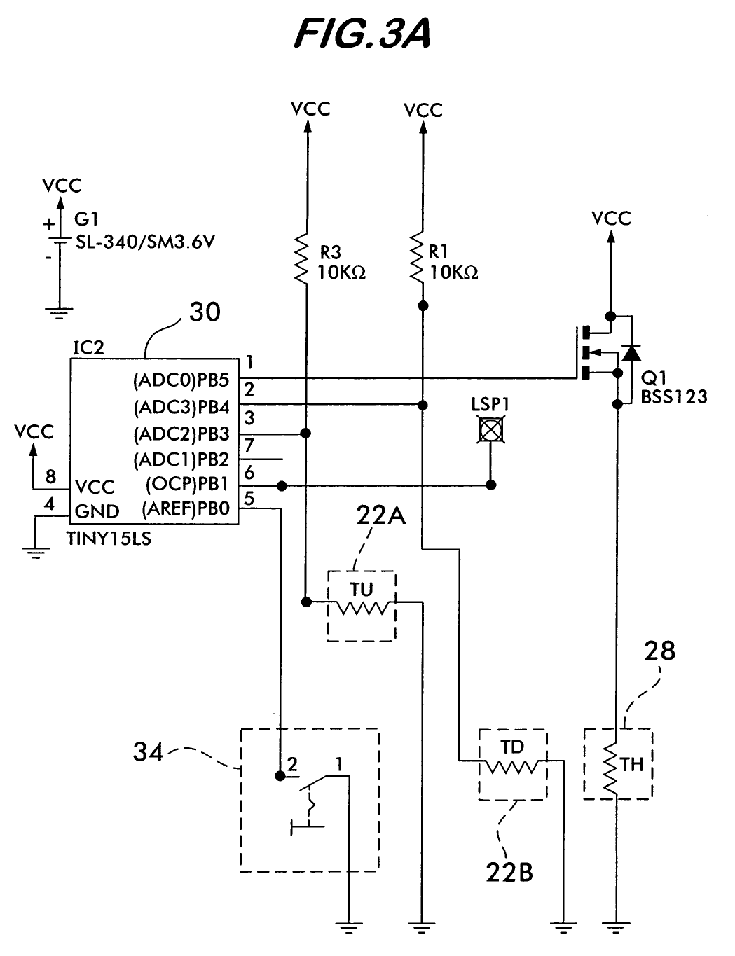

FIG. 3 depicts the microcontroller 30 and the supporting electronics 32. As can be seen from the figure, a reed switch 34, by way of example only, is coupled to the microcontroller 30 and when the remotely-located or external activator 26, e.g., a magnet, is positioned adjacent the living being LB, the wireless signal 23 (e.g., magnetic field) activates the reed switch 34 which closes, thereby changing the logic level to the microcontroller 30 which immediately pulses the heating element 28 through switch T1 (e.g., MOSFET, FIG. 3). An energy pulse (e.g., 0.6 joules) heats the fluid surrounding the heating element 28. Based on the thermal diffusion, upstream (TU/22A) and downstream (TD/22B) temperature sensors obtain temperature values and pass them onto a differential amplifier 36 that feeds the temperature difference (ΔT) to the microcontroller 30. The microcontroller 30 then uses the ΔT as discussed below. Although the “non-slip” condition dictates that no flow occurs at the fluid-wall boundary, as soon as thermal diffusion raises the temperature of the fluid radially inward of the wall, the thermal profile is affected by flow. To support the operation of the flow detector 22, the detector 22 may be powered by a lithium battery, which can be expected to last for approximately 1000 tests or 5-10 years. Other less preferable electrical power sources (including external ones) may be used.

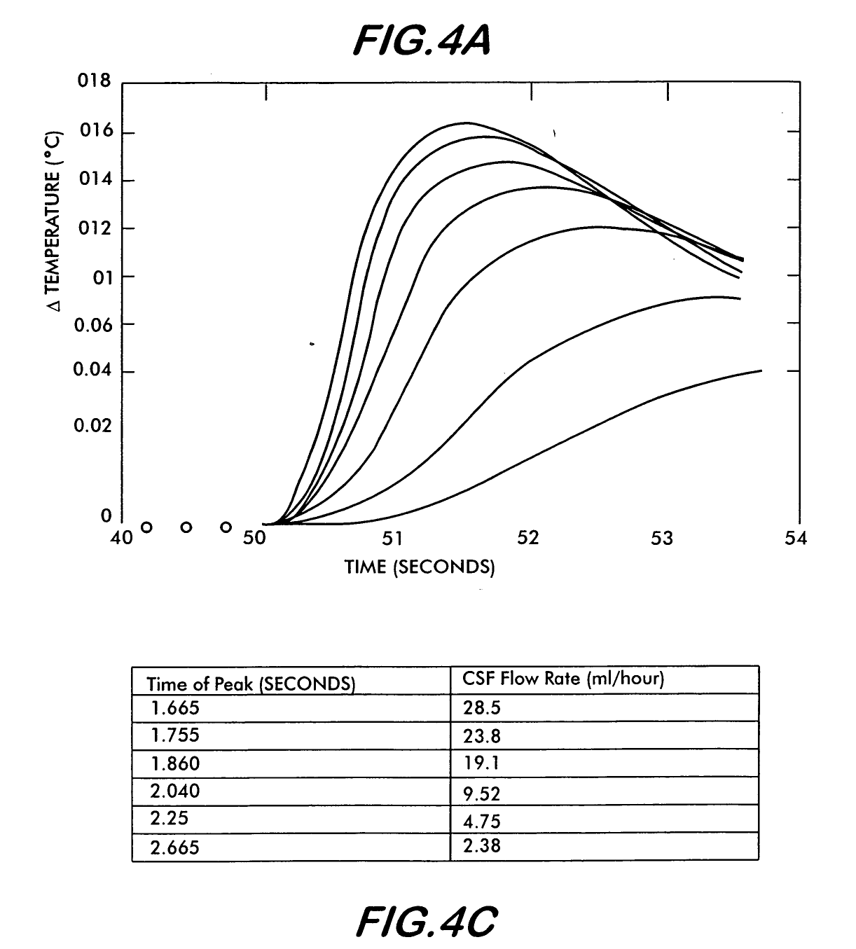

Simulation and testing of CSF flow has produced flow rate profiles as those shown in FIG. 4A. It should be noted that since the velocity of the CSF flow in typical shunt tubing is approximately 1 mm/second, CSF flow comprises a low Reynolds number and as a result, CSF flow is considered laminar flow. FIG. 4B provides test results relating peak temperature differences (which corresponds to the largest value of ΔT, i.e., the difference between the sensed temperature values of sensors 22A/22B) to corresponding CSF flow rates. In particular, as can be seen from FIG. 4A, the peak value of each plot is shifted to the right in time for slower flow rates. Thus, one exemplary mechanism for detecting CSF flow rate is to use the correspondence between the time of occurrence of the peak (based on the pulsing of the heating element 28) and the known CSF flow rate. Thus, a look-up table (FIG. 4C) has been generated that relates time of peak to a CSF flow rate. By way of example only, when the peak of the temperature profile occurs at approximately 1.665 seconds following activation of the heating element 28, the microcontroller 30, uses the look-up table to determine that such a peak occurrence corresponds to a CSF flow rate of 28.5 ml/hour. Consequently, time of peaks occurring later in time correspond to slower CSF flow rates.

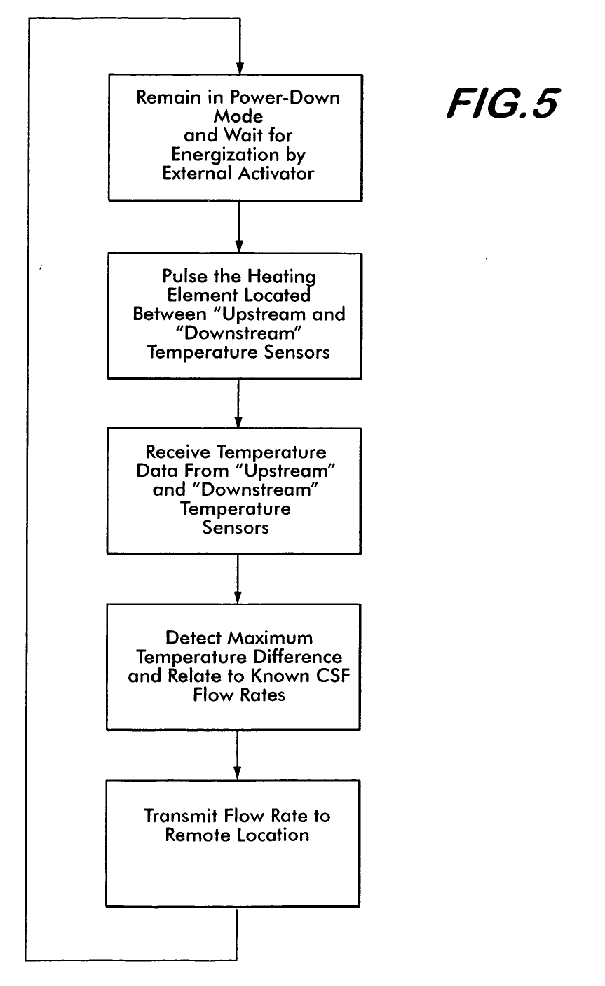

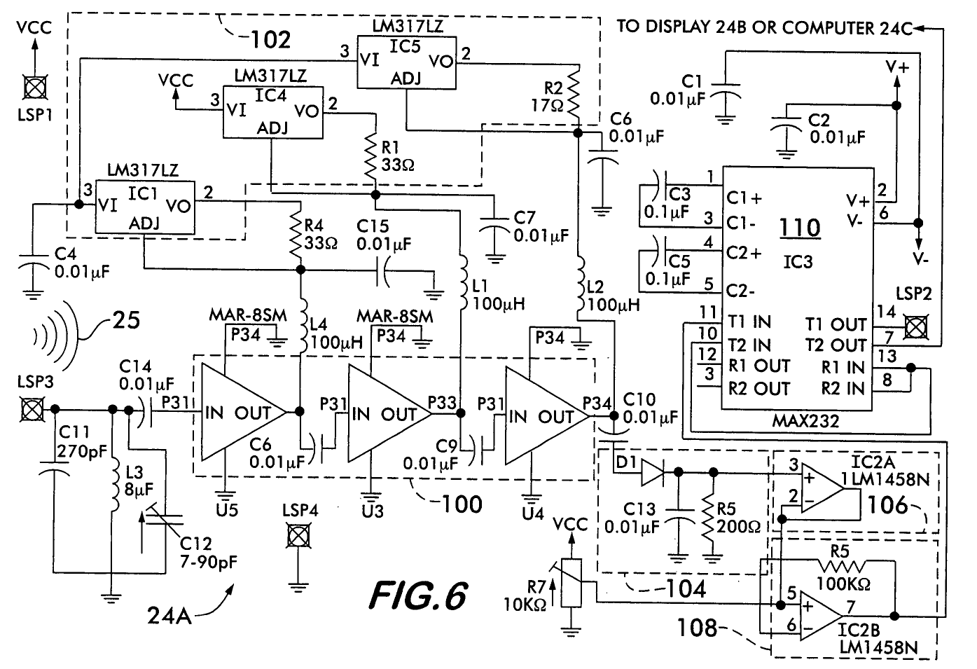

FIG. 5 depicts the microcontroller 30 operation. Most of the time, the microcontroller 30 is in a power-down (e.g., a reduced power or “sleep”) mode. When the wireless signal 23 is received, the microcontroller 30 is awakened and pulses the heating element 28. The microcontroller 30 then awaits to receive the temperature data from the temperature sensors 22A/22B. Where differential temperature (ΔT) values are provided by the supporting electronics 32, the microcontroller 30 uses that parameter (ΔT) to determine the flow rate.

By way of example only, the microcontroller 30 determines the flow by comparing the maximum temperature difference between the two thermistors 22A/22B with a table of values stored in its memory (see FIG. 4C). The microcontroller 30 then wirelessly transmits the selected CSF flow rate as 300 baud ASCII data by pulsing the on-chip PWM oscillator (150 kHz) resulting in a wireless signal 25 that is received by the detector 24A. Once the wireless signal 25 is transmitted, the microcontroller 30 returns to its power-down mode and awaits the next energization signal 23.

By way of example only, the detector 24A may comprise cascaded high gain amplifiers 100 (e.g., MMICs i.e., monolithic microwave integrated circuits, such as the Mini-Circuit MAR-8SM high gain Darlington amplifier) powered by constant current sources 102 (e.g., LM317). This is followed by a diode detector 104, then an op-amp voltage follower 106, an op-amp Schmidt trigger 108, and a voltage generator/line driver 110 (e.g., TI MAX232 which is a RS-232 voltage generator and line driver). The line driver output can displayed directly on a serial character display 24B (e.g., vacuum fluorescent display CU20029SCPB-T20A from Noritake Itron) or computer (e.g., laptop) display 24C. The term remotely-located receiver/display 24 is meant to cover any combination of a receiver and display whereby the wireless signal 25 can be detected and perceived (e.g., using a display, a speaker, an audio chip, etc.) by an individual who desires the flow rate information. Thus, the present invention is not limited, in any way, to a discrete receiver coupled to a display or computer but can include any type of integrated device or distributed device that can receive the wireless signal 25 and convert the information therein so that it can be perceived by an individual. Thus, the term “display” as used throughout this application is not limited to visual perception but includes audible perception by the individual, e.g., a speaker, an audio chip, etc. Moreover, the proximity of the display to the receiver is not required either; for example, the detector 24A may communicate over a communication link (telephone, network, fax, etc.) where the display is located hundreds of miles away from the detector 24A.

It should be understood that the manner in which the temperature data obtained by the upstream and downstream sensors 22A/22B are used by the microcontroller 30 is not limited to the manner described previously but could include using other methodologies such as integrating the area under the velocity profile, calculating a temperature difference externally of the microcontroller 30, using curve fitting, etc. For example, as shown in FIG. 3A, the supporting electronics 32 can be configured to pass absolute temperature values to the microcontroller 30 directly, instead of the difference value.

It should also be understood that the use of the magnetic reed switch is by way of example only. Other “wireless” methodologies can be used to have the wireless signal 23 activate the microcontroller 30, such as a low power radio frequency (RF) signal, ultrasonic signal, infrared (IR) signal, etc.

It should be understood that many physical arrangements of thermistors and heating element(s) are possible, wherein some may have better signal/noise ratios or some may be more suitable for certain kinds of patients. Other types of heating elements may be used, or heat may be generated by passing current through the temperature sensors themselves.

While the invention has been described in detail and with reference to specific examples thereof, it will be apparent to one skilled in the art that various changes and modifications can be made therein without departing from the spirit and scope thereof.

1. A system for detecting the flow rate of cerebrospinal fluid (CSF) in a CSF shunt implanted inside a living being, said system comprising:
a flow detector that is momentarily powered to generate a heat pulse in said CSF flow and whose movement over time therein is detected using a pair of temperature sensors, said flow detector further comprising a processor for determining a CSF flow rate from said detected movement and wirelessly transmitting a signal representative of said determined CSF flow rate;
an activator, external to the living being, that causes said flow detector to be momentarily powered; and
a remotely-located receiver for receiving said signal representative of said determined CSF flow rate.

2. The system of claim 1 wherein said flow detector further comprises a heating element for generating said heat pulse.

3. The system of claim 2 wherein said heating element is located between said pair of temperature sensors and wherein one of said temperature sensors is located upstream of said heating element and wherein the other one of said temperature sensors is located downstream of said heating element.

4. The system of claim 3 wherein each of said pair of said temperature sensors is located approximately 2 mm from said heating element.

5. The system of claim 2 wherein said heating element, when energized, provides approximately 0.6 joules of heat.

6. The system of claim 1 wherein the CSF shunt comprises a wall and wherein said flow detector is embedded within the wall.

7. The system of claim 1 wherein the CSF shunt comprises a wall and wherein said flow detector is positioned on the wall.

8. The system of claim 2 wherein said processor controls the operation of said heating element, said processor being momentarily powered by said activator to pulse said heating element.

9. The system of claim 8 wherein a magnetic reed switch controls a logic level to said processor and wherein said activator is a magnet that closes said magnetic reed switch when said activator is brought into proximity with the living being.

10. The system of claim 3 wherein said temperature sensors comprise electrical signals representative of the temperature they are detecting respectively, and wherein the difference between these signals are provided to said processor.

11. The system of claim 3 wherein said temperature sensors comprise electrical signals representative of the temperature they are detecting respectively, and wherein said electrical signals are provided to said processor, said processor determining the differences between these signals.

12. The system of claim 10 wherein said processor comprises a memory that comprises a relationship between maximum temperature differences and known CSF flow rates.

13. The system of claim 8 wherein said processor remains operational for a predetermined time after it is activated for transmitting said signal representative of said determined CSF flow rate and then said processor automatically transitions to a reduced power level.

14. The system of claim 1 wherein said remotely-located receiver includes, or is coupled to, a display for making the detected CSF flow rate perceptible to an individual.

15. A method for detecting the flow rate of cerebrospinal fluid (CSF) in a CSF shunt implanted inside a living being, said method comprising the steps of:
generating a heat pulse at a predetermined location along or within the CSF shunt;
detecting at least one temperature value of the CSF flow upstream of said predetermined location and detecting at least one temperature value of the CSF flow downstream of said predetermined location;
obtaining a maximum temperature difference value between said at least one upstream temperature value and said at least one downstream temperature value;
relating said maximum temperature difference value to a known CSF flow rate to determine the CSF flow rate; and
wirelessly transmitting said determined CSF flow rate to a remote location.

16. The method of claim 15 further comprising the step of providing said determined CSF flow rate in a form that is perceptible by an individual.

17. The method of claim 15 wherein said step of generating a heat pulse comprises embedding a heat source within the living being that is momentarily energized by a wireless signal from a device external to the living being.

18. The method of claim 17 wherein said step of detecting at least one temperature value of the CSF flow comprises disposing a first temperature sensor upstream of said heat source along or within the CSF shunt and disposing a second temperature sensor downstream of said heat source along or within the CSF shunt.

19. The method of claim 18 wherein said step of disposing a first and second temperature sensor along or within the CSF shunt comprises disposing each of said temperature sensors approximately 2 mm from said heat source.

20. The method of claim 18 wherein said step of generating a heat pulse comprises coupling said heat source to a processor and wherein said heat source is momentarily energized by momentarily energizing said processor.

21. The method of claim 20 wherein said step of disposing first and second temperature sensors comprises coupling a respective output of said first and second temperature sensors to said processor, said processor remaining momentarily energized to receive temperature data from said first and second temperature sensors to obtain said maximum temperature difference.

22. The method of claim 21 further comprising the step of said processor transitioning into a reduced power mode after wirelessly transmitting said determined CSF flow rate to a remote location.

23. The method of claim 15 wherein said step of generating a heat pulse comprises approximately 0.6 joules of heat.

24. The method of claim 15 wherein said step of relating said maximum temperature difference value to a known CSF flow rate comprises storing relationships of maximum temperature differences to known CSF flow rates in a memory in said processor.
